(19) United States
(12) Patent Application Publication
(10) Pub. No.: US 2010/0304457 A1
(43) Pub. Date: Dec. 2, 2010

(54) Method for producing biogas in controlled concentrations of trace elements

(75) Inventors: Hans-Werner Oechsner, Oberboihingen (DE); Andreas Lemmer, Stuttgart (DE); Dietmar Ramhold, Strenglin (DE); Edmund Mathies, Moorrege (DE); Elisabeth Mayrhuber, Kapfenberg (AT); Daniel Preissler, Stuttgart (DE)

Correspondence Address: VIDAS, ARRETT & STEINKRAUS, P.A., SUITE 400, 6640 SHADY OAK ROAD, EDEN PRAIRIE, MN, 55344, US

(73) Assignee: IS FORSCHUNGSGESELLSCHAFT MBH, Pinneberg (DE)

(21) Appl. No.: 12/602,045

(22) PCT Filed: May 29, 2008
(86) PCT No.: PCT/EP08/04266

§ 371 Date: Aug. 20, 2010

(30) Foreign Application Priority Data
May 29, 2007 (DE) 10 2007 025 155.8

(51) Int. Cl. C12P 5/02

(52) U.S. Cl. 435/167

(57) ABSTRACT

A method for producing biogas from biomass in a biogas reactor, wherein
at least one standard value is provided for the concentration of at least one trace element in a biogas reactor for efficient biogas production,
biogas is produced from biomass in the biogas reactor,
the concentration of at least one trace element in the biomass is determined in the biogas reactor, and
in the event that the determined trace element concentration falls below the standard value, deficient trace elements are added to the biogas reactor.

### CROSS-REFERENCE TO RELATED APPLICATIONS

Not applicable

### STATEMENT REGARDING FEDERALLY SPONSORED RESEARCH

Not applicable

### BACKGROUND OF THE INVENTION

The present invention is related to a method for producing biogas from organic mass in a biogas reactor (called also fermenter in the following).

The fixation of solar energy in biomass by the photosynthesis of plants is one of the most important sources of self-renewable energy sources (Maurer, M. and Winkler, J.-P; Biogas. Theoretische Grundlagen, Bau and Betrieb von Anlagen; 1982; edited by Springer publishing house). Based on the energy production by photosynthesis, macromolecules are synthesized by the plants as a result of metabolism. In the anaerobic degradation in biogas plants, these macromolecules can be converted to methane and carbon dioxide with a very high efficiency, so that up to 82% of the energy stored in the plants are transferred into methane.

The process of biogas production can be subdivided into four stages. In a first step, namely the hydrolysis, the complex structures of the biomass are decomposed into their monomers (sugar, fats, proteins). Subsequently there is a degradation of the monomers into short-chain fatty acids (acidogenesis). In the third (acetogenesis) and fourth step (methanogenesis), the generation of acetic acid occurs first of all, and following to this that of methane. Particularly carbon dioxide and further gases in small concentrations arise as by-products in the biogas process. The optimum environmental conditions differ partially considerably in the respective steps. (SAHM: Biologie der Methanbildung, Chem.-Ing. Tech. 53 (1981) Nr. 11, S. 854-863).

According to the state of the art, the anaerobic degradation of organic substance takes place in an aqueous medium with contents of dry substance of normally less than 30%.

The production of biogas takes place at different optimum temperatures in the range of 20 to 57° C., depending on the microorganisms involved in the process.

The optimum carbon:nitrogen:phosphorus:sulfur ratio is 500:15:5:3 for hydrolysis and acidogenesis, and 600:15:5:3 for acetogenesis and methanogenesis, respectively.

The optimum pH-value for hydrolysis and acidogenesis is in the range of pH 5.2 to 6.3, the optimum pH-value for acetogenesis and methanogenesis is in the range of pH 6.7 to 7.5.

Solid and liquid substrates are used as fermentation substrates. Both biogenic wastes from industry, trade, agriculture and households as well as energy plants purposefully grown for the production of methane are used in biogas plants. Frequently animal excreta are additionally supplied to the process in agricultural biogas plants in order to exploit their energy potential in addition. Frequently, the biogas reactor is provided with liquid manure together with the harvested energy plants at the beginning of the process of biogas production, and after that, the biogas reactor is fed exclusively with the harvested energy plants. The present invention refers to all the variants of biogas production.

The last one of the degradation steps, the generation of methane, takes place by the methanogenic microorganisms that belong to the group of archae (archaea bacteria). Together with the halo bacteria and some hyperthermophilic fermenting bacteria, they form the branch of the Euryarcheota (Schlegel, H.-G; Allgemeine Mikrobiologie; 8. ed., 2007, Georg Thieme publishing house). Among all living beings, the methanogenic ones occupy a special position. Many of their metabolic processes can proceed only with the aid of co-enzymes which only quite occasionally play a role in other microorganisms. One of the up to now known 7 is the co-enzyme F430, a cofactor with a nickel central ion. A further example is formyl-methanofuran-dehydrogenase with a molybdenum cofactor (SCHLEGEL, loc. cit. 2007). Due to these unique metabolic processes, the methanogenic organisms have special requirements regarding the concentration of trace elements.

It is already known to supply additives containing trace elements to the fermenter of biogas plants. The document EP 1 577 269 A1 discloses the addition of a zeolithe loaded with trace elements in order to compensate for a shortage of trace elements that are important for the methane gas bacteria. The fermentation substrate is for example a mixture of pig liquid manure and maize silage. When known additives with trace elements are added, only temporary, small or no improvements at all of the biogas production are achieved in part.

Starting from this, the present invention is based on the objective to provide a method for biogas production which features a significantly improved provision of the microorganisms with trace elements.

### BRIEF SUMMARY OF THE INVENTION

The method of the present invention for producing biogas from biomass in a biogas reactor comprises the following steps:
at least one standard value is provided for the concentration of at least one trace element in a biogas reactor for efficient biogas production,
biogas is produced from biomass in the biogas reactor,
the concentration of at least one trace element in the biomass in the biogas reactor is determined, and
in the event that the determined concentration of a trace element falls below the standard value of the trace element, this trace element is added to the biogas reactor.

The present invention starts from the surprising finding that the biogas production in the biogas reactor is particularly efficient when the concentration of at least one trace element that is relevant for the biogas production complies with a standard value. Relevant trace elements and standard values for their concentration in the biogas reactor have been determined by investigations with laboratory-scale plants and plants in practical use. It can be assumed that further findings will be obtained by further investigations, which permit to provide further or more accurate standard values. In the method of the present invention, the real concentration of at least one trace element is determined in the biomass in the biogas reactor (also called “fermenter content” or “fermentation substrate”). The biomass is in particular the fermentation substrates mentioned in the beginning, plus microorganisms contained therein or added to it, as the case may be. When the concentration falls below the standard value, the respective trace element is added to the biogas reactor. In doing so, the addition of the trace element can be restricted to cases where a significant shortfall from the standard value (for instance about a given tolerance) is at hand. When the real concentration of the trace element falls above the standard value (optionally minus the tolerance), the addition of the trace element is omitted. Too high concentrations of the trace elements should namely have to be avoided, because the biogas production in the biogas reactor can be damaged through this. Moreover, overdosages have the result that the areas onto which the fermentation residues are deployed are unnecessarily loaded with heavy metals. By complying with the standard value of at least one trace element, a more efficient biogas production is achieved by doing so. Preferably, the observance of the standard values is monitored for plural trace elements, and if necessary made sure by the addition of trace elements. Thus, the trace element addition serves for the stabilisation and output increase of the methane gas production from organic substance. When a trace element shortage in the fermentation substrate is compensated, the population density and the performance of the biologic matter contained in the fermenter is increased, and thus, an increase of the substrate turnover in the biogas plant is made possible.

The investigations have shown that the control of the compliance with standard values of certain trace elements is especially important for the effectiveness of the biogas production. In that, it is dealt with the trace elements nickel, cobalt, molybdenum and iron. Therefore, according to an embodiment of the method, standard values are provided for the concentrations of the trace elements nickel and/or cobalt and/or molybdenum and/or iron and the concentrations of the trace elements nickel and/or cobalt and/or molybdenum and/or iron in the biomass in the biogas reactor are determined. A possible shortage of the mentioned trace elements in the biogas reactor can then be compensated.

According to a further embodiment, the standard values for nickel are 4 to 30 mg/kg DM and/or for cobalt 0.4 to 10 mg/kg DM and/or for molybdenum 0.05 to 16 mg/kg DM and/or for iron 750 to 5000 mg/kg DM.

According to a further embodiment, the standard values for nickel are at least 10 and/or at most 25 mg/kg DM and/or for cobalt at least 1.0 and/or at most 5.0 mg/kg DM and/or for molybdenum at least 1.0 and/or at most 10.0 mg/kg DM and/or for iron at least 1500 and/or at most 3500 mg/kg DM.

According to the present state of research, the optimal standard values for nickel are 16 mg/kg DS and/or for cobalt 1.8 mg/kg DS and/or for molybedenum 4 mg/kg DS and/or for iron 2400 mg/kg DS.

The investigations have further shown that also other trace elements are of importance in the biogas production. The trace elements in question are manganese, copper, selenium, tungsten and zinc. According to an embodiment of the procedure, standard values are therefore provided for the concentration of the trace elements manganese and/or copper and/or selenium and/or tungsten and/or zinc, and the concentrations of the trace elements manganese and/or copper and/or selenium and/or tungsten and/or zinc in the biogas reactor are determined. In the case of a shortage, the respective trace element is added to the biogas reactor.

According to a further embodiment, the standard values for manganese are 100 to 1500 mg/kg DM and/or for copper 10 to 80 mg/kg DM and/or for selenium 0.05 to 4 mg/kg DM and/or for tungsten 0.1 to 30 mg/kg DM and/or for zinc 30 to 400 mg/kg DM.

According to a further embodiment, the standard values for manganese are at least 250 and/or at most 350 mg/kg DM and/or for copper at least 30 and/or at most 50 mg/kg DM and/or for selenium at least 0.3 and/or at most 0.7 mg/kg DM and/or for tungsten at least 0.4 and/or at most 0.8 mg/kg DM and/or for zinc at least 150 and/or at most 250 mg/kg DM.

According to the present state of research, the optimal concentrations are for manganese 300 mg/kg DM and/or for copper 40 DM mg/kg and/or for selenium 0.5 DM mg/kg and/or for tungsten 0.6 DM mg/kg and/or for zinc 200 DM mg/kg.

A compensation of a shortage of trace elements should occur considering the biological availability and the actual need. According to an embodiment of the method, the availability of the trace elements contained already in the fermentation substrate is increased first of all. This can occur for example through change of physical parameters of the method, like temperature, pressure, dry matter proportion, water content, mixing intensity. According to an embodiment, the biogas reactor is provided with an additive that increases the biological availability of the trace elements. The biological availability of the trace elements is reduced through high sulphide concentration; hardly soluble and not biologically available metal sulphides precipitate. According to an embodiment of the method, the biological availability is increased by addition of an agent that reduces the sulphide concentration. Due to the good affinity of iron to sulphide, the sulphide ions can be fixed by iron addition, so that trace elements provided only in small amounts are fixed through the sulphides in a smaller extent. In this it is a favourable effect that iron does not lead to inhibition of the biogas production in the fermenter, not even at high concentrations. Therefore, the trace element iron is added to the biogas reactor according to an embodiment of the method.

According to a further embodiment of the method, the availability of the trace elements already contained in the fermentation substrate is increased first of all, and a shortage is compensated after that through addition of trace elements. A direct decrease of the biological availability of the trace elements added for shortage compensation—for example through fixation on the sulphides—is avoided by this.

According to a further embodiment of the method, the concentration of at least one trace element in the biological material is determined after the increasing of the biological availability of the trace elements, and a shortage of the trace element is compensated by adding the same. A better use of the trace elements contained in the fermentation substrate and the approach to optimal concentrations of the trace elements in the biomass are favoured by that.

The concentration of the at least one trace element in the biogas reactor can be determined in different ways. According to an embodiment of the method, the concentration is determined by ICP (inductive coupled plasma)-analysis of at least one sample from the biogas reactor.

In principle, the concentration of the at least one trace element must be determined only once in order to check the compliance with the associated standard value and to add the corresponding trace element where appropriate. The trace element concentrations within the fermenter are dependent on the respective supplied substrates and can therefore change with the feeding of the fermenter. Further, the biological availability of the trace elements can be influenced by the added substrates and process aids, and can therefore change in the course of time. According to an embodiment of the method, the concentration of at least one trace element in the biogas reactor is repeatedly determined in time intervals in order to acquire changes of the concentrations of the trace elements in the biogas reactor. The respective actual concentration of the at least one trace element is compared with the related standard value and made the basis of an actual calculation of the addition amount.

The amount of the trace elements to be added can be determined in different ways. For example, in the case of a shortage of a trace element, a given amount of the trace element can be added one-time or repeatedly in intervals. The concentration of the trace element can be determined in a time interval in the biogas reactor. Due to the determined concentration it can be found out whether a renewed addition of the given one or a differing amount is necessary. If the standard value is still fallen below, the given addition can be increased according to the proportion) of the standard value to the measured actual concentration. If the standard value is exceeded, the given addition can be reduced according to the proportion of the standard value to the measured actual concentration. In this way, an optimization of the amount to be added is possible.

According to another embodiment, a given amount of the trace element is not added at the beginning. Rather, the amount of trace elements to be added is determined depending on the difference between the standard value and the determined concentration. In the case of a great difference, a correspondingly great amount of the trace elements is added in time intervals, and in the case of a small difference a correspondingly small amount of the trace elements is added in time intervals. According to a further embodiment, in order to compensate for losses of the trace elements, the amount of trace elements to be added is determined taking into account the trace elements that were taken out of the biogas reactor with the fermentation residues.

According to an embodiment, the biogas reactor is provided once with an amount of trace elements which is dimensioned such that an immediate increase occurs to the final level of the trace elements. The addition can be repeated in intervals. In particular, it can be given into the biogas reactor anew after the decay of a part of the residence time or for instance after the residence time is ended.

According to a further embodiment, an amount of trace elements which is smaller than the need is added into the biogas reactor at the beginning. The addition is later adapted to the need. Through that, the microbiological system in the biogas reactor can gradually adapt itself to the new conditions.

In each case, the need in accordance to the period of time for which the addition occurs has to be made the basis. The period of time in which an amount of trace elements falling below the need is added is preferably smaller than the residence time of the fermentation substrate in the biogas reactor, which is for example 1 to 3 months. According to an embodiment, only a part of the amount of trace elements that has to be added is added initially within one to two weeks.

According to a further embodiment, the trace elements are put into the biogas reactor in a well soluble form. According to a further embodiment, they are distributed uniformly in the biogas reactor. Through that, an excess- and shortage situation can be avoided in the individual zones of the biogas reactor.

According to an embodiment, the trace elements are added continuously or one-time or repeatedly (for example in equal or different intervals of time and/or in equal or different amounts). For example, they are added through one-time or repeated addition of a depot which releases trace elements over a longer period of time. A one-time addition of trace elements can occur for example in order to raise the biogas production in the biogas reactor at short notice. On a long-term basis, the biogas production can then be kept on a high level by a changed feeding with biomass. A continuous or repeated addition of trace elements can occur for example if a trace element shortage of the fed biomass must be compensated on a long-term basis.

The addition of the trace elements can occur in different time intervals. According to one embodiment of the method, it occurs daily or at intervals of several days. According to another embodiment, it occurs in intervals which approximately correspond to the residence time (for example 1 to 3 months) of the biomass in the biogas reactor. These intervals are preferably the maximum intervals between the additions, because it can be assumed that the added trace elements are substantially consumed within the residence time and/or taken out of the fermenter. An addition in changing intervals is also possible.

If the individual process steps of the biogas process occur in spatially separated receptacles or biogas reactors, respectively, the different needs of the bacterium types present in the individual biogas reactors can be taken into account by the respective addition.

According to an embodiment, an additive containing different trace elements is added to the biogas reactor. The additive is for example a mixture of the different trace elements in liquid or solid form, wherein a solid additive can be added in the form of a powder or in the form of a granulate or of at least one other solid that quickly or gradually falls into parts in the fermentation substrate or is dissolved in that or releases trace elements, respectively.

According to an embodiment, the additive is specially made depending on the standard values and the determined concentrations. Thus, an additive adapted specially to need is added to the biogas reactor indeed, namely continuously, one-time or repeatedly.

According to another embodiment, additives comprising several trace elements in different amount ratios of the trace elements are made, and from these additives that one is supplied to the biogas reactor whose composition at most approaches the composition of the additive that should be added to the biogas reactor, which was determined with the aid of the standard values and the determined concentrations. In the case of this variant of the method, different standard additives are kept at hand, amongst which that one is selected in the case of need which is best suited for the compensation of a shortage of trace elements in the biogas reactor. This selected additive is added to the biogas reactor continuously, one-time or repeatedly.

### BRIEF DESCRIPTION OF FIGURES

FIG. 1, referred to as Diagram, shows the Fos/Tac value; and

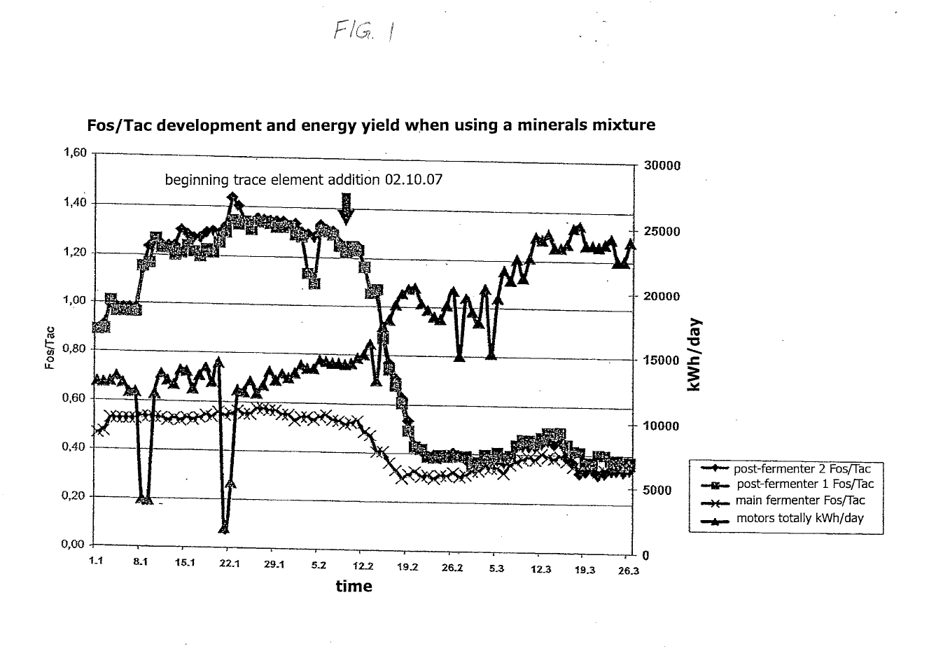

FIG. 2, referred to as the Drawing, shows a biogas plant schematically.

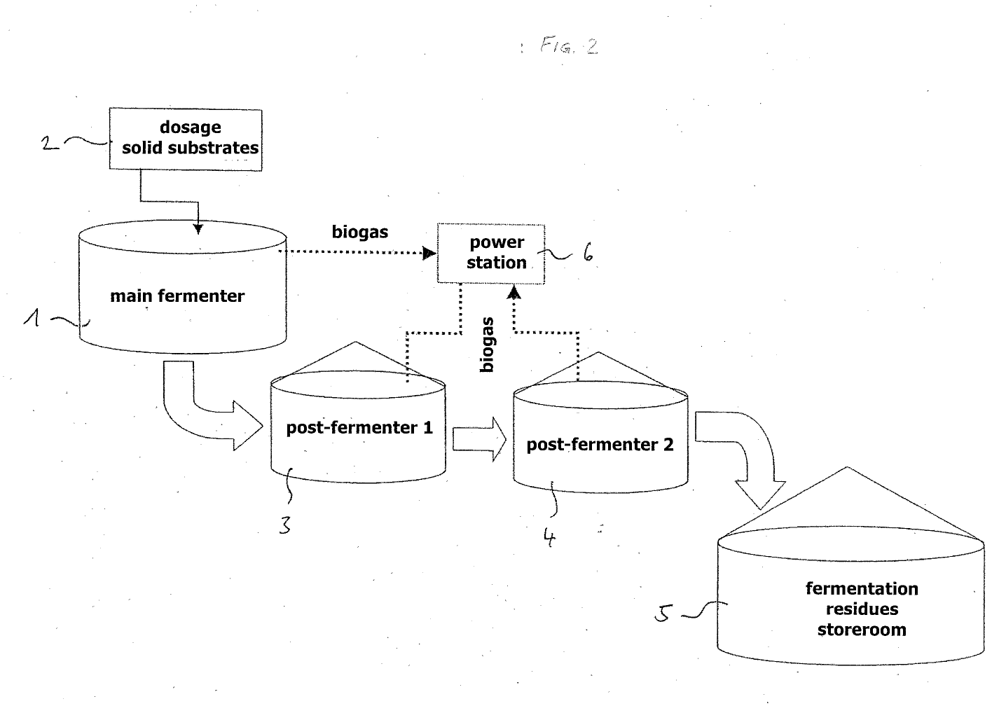

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated.

The method of analysis of the trace elements by means of ICP-analysis is explained in more detail in the following:

### Sampling:

A homogeneous sample is taken out of the fermenter that is to be examined, so that the composition in the sample is identical with the overall composition of the fermenter contents. The amount of the sample should be about 2 kg in total.

Sufficient mixing (homogeneousness) is to be provided in each processing step of the sample.

### Sample Processing:

About 600 g of the sample are weighed out into an aluminium dish that is covered with baking paper, and these are then dried for at least 48 hours at 65° C. in a circulating air oven. The sample from the fermenter is dried first of all at 65° C. in order to obtain a material which permits to be stored and to be processed. The loss of weight is acquired by weighing the sample vessel as well as the weighted-in quantity of the sample before and after drying.

### Calculation of the 65° C.-Dry Matter (in a Word DM) in %:

% DM(65° C.)=sample weight after drying/sample weight before drying×100%

The entire dry sample material is grind in a mill (fineness 1 mm sieve passage).

The material dried at 65° C. still contains certain remaining quantities of water. From the material dried at 65° C. and then milled, a determination of the dry matter is carried out at 105° C. by determining the loss of weight after 4 hours of drying at 105° C.

### Calculation of the 105° C.-DM in %:

% DM(105° C.)=sample weight after drying/sample weight before drying×100%

The remaining water content is the difference of % DM(105° C.) to 100%.

### Calculation of the Entire Dry Matter in the Fermenter:

% DM_{fermenter}=% DM(105° C.)×% DM(65° C.)/100%

### Sample Digestion:

Exactly 3 g of the homogeneous sample material are weighed out into a small quartz tube and heated up on a heating plate so strongly that the organic material begins to carbonize. As soon as the sample does not smoke any more, the small quartz tube comes into a muffle furnace to incinerate there for at least 32 hours at 550° C.

Into the small quartz tube cooled down, one adds 5 ml 65% nitric acid, as well as 0.5 ml 30% hydrogen peroxide solution and puts the small quartz tube into a microwave pressure vessel, in order to digest the sample subsequently in the microwave. The conditions of the microwave digestion are to be chosen such that a maximum amount of trace elements go into solution (approx. 7.5 min at 600 watts).

The digested sample is transferred with deionised water into a volumetric flask, normally a volumetric flask, and filled up to the measuring mark.

Measurement of the Elements by Means of ICP-Spectrometer:

Possibly existing undissolved components are filtered out and the solution is then measured by means of an ICP-OES spectrometer. ICP-OES means inductively coupled plasma with evaluation of the optical emission spectrum. This is a usual method of measurement for the determination of dissolved elements, wherein the sample solution is pumped into an approx. 5000-8000° Kelvin hot flame (produced by inductively coupled plasma). The elements contained in the test solution then emit the spectrum lines which are typical for every element and which can be processed optically and read out. The device has a calibration that had been established by means of different standard solutions with the elements that are very similar to the matrix of the fermenter contents. With the aid of the calibration, the content for each element is calculated quantitatively.

The following elements are quantitatively examined:

Sodium, calcium, potassium, magnesium, sulphur, phosphorus, copper, boron, manganese, zinc, nickel, cobalt, molybdenum, selenium, iron, tungsten.

In the future, it might also be conceivable to capture the content of further elements, provided that a relationship between the concentration of the element and the function of the fermenter is expected.

### Calculation of the Element Parts in DM:

By means of the ICP analysis, one obtains the content in mg/l for the examined elements and converts this to the content in the dry matter, considering the weight-in quantity, the dilutions and the content of remaining humidity. Thus, one obtains the content in the fermenter sludge for every examined trace element (general ME) with reference to the dry matter:

Conc. (Me)_{fermenter} in mg/kgDM

Explanation of the calculation of the addition amounts of trace elements for an optimal operation of the biogas plants

### General:

With the aid of the determined contents of the different trace elements and the knowledge which contents are necessary for an optimal biogas process, it can be calculated for each individual element whether the content of the respective trace element is sufficiently available or whether there is a deficit. When there is a deficit, this deficit must be compensated by adding well soluble and highly available trace elements as salts. A good homogeneous distribution of the trace element additives must be guaranteed in the fermenter.

Me stands generally for all trace elements. The following calculation must be carried out individually for all necessary trace elements.

### Calculation of the Deficit:

Conc.(Me)ₒₚₜᵢₘᵤₘ−Conc.(Me)_{fermenter}=deficit_{Me}(mg/kgDM)

Conc. (Me)ₒₚₜᵢₘᵤₘ in mg/kg DM=optimum concentration of the trace element Me
Conc. (Me)_{fermenter} in mg/kg DM=determined concentration of the trace element Me
When the deficit is negative, that is to say conc. (Me)ₒₚₜᵢₘᵤₘ<Conc. (Me)_{fermenter}, no addition is necessary.
When the deficit is positive, that is to say Conc. (Me)ₒₚₜᵢₘᵤₘ>Conc. (Me)_{fermenter}, addition is necessary.

### Calculation of Deficit-Compensation:

When a positive deficit was determined for a trace element, this deficit must be compensated for by addition. The compensation is calculated for the half of the actual deficit and added distributed over 7 days, so that the microbiological system can slowly adapt itself to the new conditions. For the determination, it can be conveniently assumed that the fermenter content in (m³) is equal to the mass in (to).

### Trace Element Addition in 7 Days for 50% Compensation of the Deficit:

Fermenter content(to)×DM_{fermenter} (%)×deficit_{Me} (mg/kgDM)×0.5/100%=Addition_{Me50% desired}(g)

Since the trace element is used in the form of a salt or a salt batch, the addition of the trace element must be converted into the addition of the trace element salt by considering the content of the trace element in the salt or the salt batch (% Me content of the salt).
Trace Element Salt Addition in 7 days for 50%-Compensation of the Deficit:

Addition_{Me 50% desired}(g)/% Me content of the salt×100%=addition_{Me salt 50% desired}(g)

### Discharge Loss Calculation:

After the 7 days, that amount of trace elements is added which is the daily loss of trace elements through the discharge from the fermenter and is not compensated by substrate feeding. In the case of unchanged substrate feeding over a period of several days, this daily discharge leads exactly to the deficit of trace elements mentioned at the beginning.

The calculation is performed via the hydraulic residence time (HRT) in the fermenter, which indicates how long an added substance remains in the fermenter on the average. Since only 50% of the deficit were compensated in the first 7 days, but now it is assumed that the entire deficit is discharged proportionally, it is achieved that the concentration of the trace element slowly approaches the optimal need.

### Daily Trace Element Addition for Compensation of the DISCHARGE Losses:

Fermenter content(to)×DM_{fermenter}(%)×deficit_{Me}(mg/kgDM)/100%/HRT(d)=Addition_{Me daily}(g)

Since the trace element is used in the form of a salt or a salt batch, the addition of the trace element must be converted into the addition of the trace element salt by considering the content of the trace element in the salt or the salt batch (% Me content of the salt).

### Daily Trace Element Salt Addition for Compensation of the Discharge Losses:

Addition_{Me daily}(g)/% Me content of salt×100% Addition_{Me salt daily}(g)

### Example Calculation:

In order to clarify the concrete procedure, an example is calculated by means of the trace element nickel.

Assumptions for the example:
Conc. (Ni)_{fermenter}=4,3,3 mg/kgDM according to analysis of the fermenter
Fermenter content=2.500 m³ or 2.500 to, respectively
Average residence time (HRT)=63 days
DM fermenter=8.7%
Addition as nickel sulphate hexahydrate with 22.35% nickel content

### Calculation of the Deficit:

For nickel, 4-30 mg/kg DM have been evaluated as optimal

Conc. (Ni)ₒₚₜᵢₘᵤₘ=16.0 mg/kg DM=optimum concentration of the trace element Ni

Conc.(Me)ₒₚₜᵢₘᵤₘ−Conc.(Me)_{fermenter}=deficit_{Me}(mg/kgDM)

16.0−4.3=11.7 mg/kgDM=deficit_{Ni}
The deficit is positive, that is to say Conc. (Ni)ₒₚₜᵢₘᵤₘ optimum>Conc. (Ni)_{fermenter} thus addition is necessary.

### Calculation of Deficit Compensation:

Trace element addition in 7 days for 50% compensation of the deficit:

Fermenter content(to)×% DM_{fermenter}(%)×deficit_{Me}(mg/kgDM)×0.5/100%=addition_{Me50% desired}(g)

2.500to×8.7%×11.7 mg/kgDM×0.5/100%=1272.5 g of nickel=addition_{Me 50% desired}(g)

Trace element salt addition in 7 days for 50%-compensation of the deficit:

Addition_{Me 50% desired}(g)/% Me content of the salt×100%=addition_{Me salt 50% desired}(g)

1272.5 g Ni/22.35 Ni in the salt×100%=5693.4 g of nickel sulphate hexahydrate=addition_{Me salt 50% desired}

### Discharge Loss Calculation:

Daily Trace Element Addition for Compensation of the Discharge Losses:

Fermenter content(to)×% DM_{fermenter}(%)×deficit_{Me}(mg/kgDM)/100%/HRT(d)=addition_{Me daily}(g)

2.500to×8.7%×11.7 mg/kgDM/100%/63d=40.4 g Ni=Addition_{Me daily}

### Daily Trace Element Salt Addition for Compensation of the Discharge Losses:

Addition_{Me daily}(g)/% Me content salt×100%=addition_{Me salt daily}(g)

40.4 g/22.35×100%=108.8 g of nickel sulphate hexahydrate=addition_{Me salt daily}

### Trace Element Mixture Calculation:

Because every trace element which is in deficit is should be added, a trace element mixture that contains the necessary trace elements in the relation as they were calculated from the addition amounts is calculated from the different trace element salts. An addition recommendation is calculated by means of the operating data of the biogas operator, so that the calculated addition amounts are reached. Where appropriate, a filling material is added in order to achieve a better handling suitability of the trace element mixture.

### Trace Element Mixture Addition in 7 Days for 50% Compensation of the Deficit:

Sum of all additions_{Me-salt 50% desired}(g)+filling material(g)=addition_{ΣMe mixture 50% deficit} over 7 days

For a uniform distribution over 7 days, the amount must be divided by 7 days:

### Daily Addition of Trace Element Mixture Over 7 Days for 50% Compensation of the Deficit:

Addition_{ΣMe mixture 50% deficit} over 7 days/7 days=addition_{ΣMe mixture 50% deficit} daily

An analogous mode is applied to the addition for the compensation of the discharge losses:

### Daily Addition of Trace Element Mixture for the Compensation of Discharge Losses:

Sum of all additions_{Me salt daily}(g)+filler(g)=addition_{ΣMe mixture daily}

### Results of Practice Investigations

### Example 1

A biogas plant operated free of liquid manure, that exhibited a process inhibition already since four months, with strongly increased acid values and Fos/Tac values (describing the ratio of volatile organic acids and the inorganic carbon as a measure for the buffer capacity) as well as with a consequently reduced gas production, was charged with a trace element gift which was specially adapted to this biogas plant. The feed consisted of maize silage, cereal grains and grass silage. After addition of the trace elements, both a rise of the gas quality and of the generated amount of gas occurred within 24-72 h, due to a decomposition of the acids that had accumulated due to the process inhibition before. In spite of a subsequently increased feed, the analytical values of the fermentation substrate showed a steady improvement of the process conditions. The acids reduced subsequently from formerly critical concentrations, indicating a process inhibition, to extremely low contents which evidence a stable process. As a whole, the power of the biogas increased from 600 kW to 840 kW within the first 10 days, which corresponds to an increase in performance of 40%.

The development of the Fos/Tac-values and of the energy yield before and after the application of a trace element addition are shown in the attached diagram. Here, the course of the Fos/Tac-values over time is shown in the main fermenter (x), in the post-fermenter 1 (squares) and in the post-fermenter 2 (lozenges). Further, the overall power of the motors (triangles) is also shown. The respective measured values are connected through curves. It can be recognised easily that the performance of the biogas plant increases about 40% within 10 days after the trace element addition.

The following is to be said about the Fos/Tac-values:

The Fos/Tac value has proven to be of value in the analysis of biogas fermenters and is performed in virtually all investigations.

The sum of the organic acids (Fos) and the sum of the carbonate buffer (Tac) can be determined by titration with a certain acid.

The ration Fos/Tac resulting from this should be below 0.3, which means that the ratio between buffer and acid is balanced.

If the value increases above 0.4, there are too much acids for the carbonate buffer at hand. This is an unambiguous, well known indication of a not optimal biogas process, frequently triggered in that the acids are not degraded fast enough or not sufficiently.

20 ml of a centrifugated fermenter sample are diluted with approx. 80 ml of water, and during agitation, it is titrated with 0.1n sulphuric acid and the pH-value is measured during this.

One lists the consumption of sulphuric acid (ml 0.1n sulphuric acid) up to the pH-value 5.0 (=α) and continues to titrate up to the pH-value 4.4. One lists the consumption of sulphuric acid (ml 0.1n sulphuric acid) from pH 5.0 up to pH 4.4 (=β).

Tac=α×250

Fos=(β×1.66-0.15)×500

Fos/Tac=Fos:Tac

### Example 2

In a biogas plant operated in co-fermentation of bovine liquid manure, Sudan grass and wheat grain, only digestion tank loads of 2 kg of organic substance per cubic meter fermenter volume were realizable. When the feed was raised, the short-chain fatty acids accumulated which are normally degraded to methane and carbon dioxide in further steps, and there was an inhibition of the decomposition with imminent breakdown of the biogas generation. The biogas plant has two identical fermenters, which were equally loaded. One of these fermenters was treated with trace elements, the second was operated as before as a control. After the trace element treatment, there was a rapid increase of the biogas amount and quality, whereas the untreated fermenter showed no changes. The increased gas amount resulted from a decomposition of the organic acids, which could now be decomposed to the final products methane and carbon dioxide, due to the now no more inhibited biological activity (Table 1). A subsequent raise of the supply of organic substance resulted in an increased gas production, but without further signs of an inhibition. The fermenter kept on being operated without trace element addition as a control showed only a small improvement of the analytical values, in spite of a significantly lower load.

TABLE 1
Development of the volatile fatty acids of a biogas plant after trace element
addition, compared with the control variant (target values of a stable biogas process:
ratio of acetic acid to propionic acid >2:1, propionic acid <1000 mg/kg FM).

| | fermenter 1 | | | fermenter 2 | | |
|---|---|---|---|---|---|---|
| | acetate | propionate | butyrate | acetate | propionate | butyrate |
| | [mg/ | [mg/kg | [mg/kg | [mg/kg | [mg/kg | [mg/kg |
| date | kg FM] | FM] | FM] | FM] | FM] | FM] |
| Feb. 20, 2007 | 1.385 | 4.470 | 701 | 1.055 | 4.484 | 586 |
| Feb. 20, 2007 | addition of the trace elements | | | no addition of the trace elements | | |
| Mar. 05, 2007 | 679 | 1216 | 370 | 1.1016 | 3.805 | 529 |
| Mar. 12, 2007 | 203 | 76 | 2 | 738 | 3.109 | 455 |

### Trace Element Supply of Plant 1

| | starting concentration | addition amount |
|---|---|---|
| element | [mg/kg DS] | [mg/kg DS] |
| nickel | 2.3 | 14.2 |
| cobalt | 0.5 | 0.3 |
| molybdenum | 1.5 | 1.3 |
| iron | 826 | 769 |
| manganese | 131 | No addition |
| copper | 19.3 | No addition |
| selenium | 0.22 | No addition |
| tungsten | not acquired | No addition |
| zinc | 138 | No addition |

The standard values of the concentrations of the trace elements provided according to the present invention, as well as their optimum range and the limit values for the deposition on agricultural areas, are summarised in the following overview:

### Standard Values of the Optimum Trace Element Concentrations

| | optimum range | desired range | limit values |
|---|---|---|---|
| element | [mg/kg DS] | [mg/kg DS] | [mg/kg DS] |
| nickel | 16 | 4-30 | 50 (30)*) |
| cobalt | 1.8 | 0.4-10 | |
| molybdenum | 4 | 0.05-16 | |
| iron | 2400 | 750-5000 | |
| manganese | 300 | 100-1500 | |
| copper | 40 | 10-80 | 100*) |
| selenium | 0.5 | 0.05-4 | |
| tungsten | 0.6 | 0.1-30 | |
| zinc | 200 | 30-400 | 400*) |

*1) limit values of the German regulation (BioAbfV) for deposition on agricultural areas,
in parentheses: regulation concerning environment compromising substances (Stoffverordnung StoV), modification of mart 26 2003 in the name of the Swiss government

Standard values fall always significantly below the limit values if such values exist.

In the drawing, a biogas plant is shown in a rough, schematic manner, to which trace elements can be supplied according to the present invention in order to compensate a shortage of trace elements.

The biogas plant comprises a main fermenter 1, into which solid substrates can be metered via a dosage apparatus 2. Behind the main fermenter is connected a post-fermenter 3, and behind the latter is in turn connected a further post-fermenter 4. From the further post-fermenter 4, fermentation residues reach a fermentation residue storage room 5.

From the main fermenter 1, the post fermenter 3 and the further post-fermenter 4, the biogases are supplied to a block-type thermal power station 6, which produces electrical current and heat for warming up rooms.

In the main fermenter 1 occurs a part of the biogas production, from the hydrolysis up to the methane generation. Also, most of the biogas is drawn out here. A residual methane generation, accompanied by further degradation of the biomass, takes place in the post-fermenters 3 and 4. A shortage of trace elements is compensated by supplying trace elements to the biogas plant via the dosage apparatus 2 for fine substrates.

This completes the description of the preferred and alternate embodiments of the invention. Those skilled in the art may recognize other equivalents to the specific embodiment described herein which equivalents are intended to be encompassed by the claims attached hereto.

1. A method for producing biogas from biomass in a biogas reactor, wherein
at least one standard value is provided for the concentration of at least one trace element in a biogas reactor for efficient biogas production,
biogas is produced from biomass in the biogas reactor,
the concentration of at least one trace element in the biomass is determined in the biogas reactor, and
in the event that the determined trace element concentration falls below the standard value, deficient trace elements are added to the biogas reactor:
the standard values for nickel are 4 to 30 mg/kg DM and/or for cobalt 0.4 to 10 mg/kg DM and/or for molybdenum 0.05 to 16 mg/kg DM and/or for iron 750 to 5000 mg/kg DM.

2. A method according to claim 1, wherein standard values are provided for the concentration of the trace elements nickel and/or cobalt and/or molybdenum and/or iron, and the concentration of the trace elements nickel and/or cobalt and/or molybdenum and/or iron is determined in the biogas reactor.

3. A method according to claim 1, wherein the standard values for nickel are at least 10 and/or at most 25 mg/kg DM and/or for cobalt at least 1.0 and/or at most 5.0 mg/kg DM and/or for molybdenum at least 1.0 and/or at most 10.0 mg/kg DM and/or for iron at least 1500 and/or at most 3500 mg/kg DM.

4. A method according to claim 1, wherein standard values are provided for the concentrations of the trace elements manganese and/or copper and/or selenium and/or tungsten and/or zinc, and the concentrations of the trace elements manganese and/or copper and/or selenium and/or tungsten and/or zinc in the biogas reactor are determined.

5. A method according to claim 4, wherein the standard values for manganese are 100 to 1500 mg/kg DM and/or for copper 10 to 80 mg/kg DM and/or for selenium 0.05 to 4 mg/kg DM and/or for tungsten 0.1 to 30 mg/kg DM and/or for zinc 30 to 400 mg/kg DM.

6. A method according to claim 5, wherein the standard values for manganese are at least 250 and/or at most 350 mg/kg DM and/or for copper at least 30 and/or at most 50 mg/kg DM and/or for selenium at least 0.3 and/or at most 0.7 mg/kg DM and/or for tungsten at least 0.4 and/or at most 0.8 mg/kg DM and/or for zinc at least 150 and/or at most 250 mg/kg DM.

7. A method according to claim 1, wherein the biological availability of the trace elements that are contained in the biomaterial in the biogas reactor is increased.

8. A method according to claim 7, wherein an additive increasing the biological availability of the trace elements is supplied to the biogas reactor.

9. A method according to claim 8, wherein the additive contains iron.

10. A method according to claim 7, wherein at least one trace element is added after the increasing of the biological availability of the trace elements.

11. A method according to claim 7, wherein the concentration of at least one trace element in the biological material is determined after the increasing of the biological availability of the trace elements, and a shortage of the trace element is compensated by adding the same.

12. A method according to claim 1, wherein the concentration of at least one trace element in at least one sample from the biogas reactor is determined by ICP analysis.

13. A method according to claim 1 wherein the concentration of at least one trace element in the biogas reactor is repeatedly determined in time intervals.

14. A method according to claim 1 wherein the amount of trace elements to be added is determined depending on the difference between the standard value and the determined concentration.

15. A method according to claim 14, wherein the amount of trace elements to be added is determined taking into account the trace elements that were taken out of the biogas reactor with the fermentation residues.

16. A method according to claim 1 wherein only a part of the amount of trace elements to be added is added initially, and amounts corresponding to the need of trace elements to be added are added later.

17. A method according to claim 16, wherein a part of the amount of trace elements to be added is added initially within one to two weeks.

18. A method according to claim 1, wherein the trace elements are added continuously or one-time or repeatedly, and/or are added by one-time or repeated addition of a depot which releases trace elements over a longer period of time.

19. A method according to claim 1, wherein an additive comprising different trace elements is added to the biogas reactor.

20. A method according to claim 19, wherein the additive is specially produced depending on the standard values and the determined concentrations.

21. A method according to claim 19, wherein additives comprising plural different trace elements in different amount ratios of the trace elements are produced, and that one of these additives is supplied to the biogas reactor whose composition most approaches the composition of the additive to be added to the biogas reactor that was determined with the aid of the standard values and the determined concentrations.
